(19) United States
(12) Patent Application Publication
(10) Pub. No.: US 2005/0118664 A1
(43) Pub. Date: Jun. 2, 2005

(54) Fluorogenic protease substrates

(76) Inventors: Michael John Blackman, Hertfordshire (GB); Thomas Corrie, Hertfordshire (GB); John Frederick Eccleston, Hertfordshire (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/501,131

(22) PCT Filed: Jan. 9, 2003
(86) PCT No.: PCT/GB03/00045

(30) Foreign Application Priority Data
Jan. 9, 2002 (GB) 0200479.4

(51) Int. Cl. C12Q 1/37; C07K 7/00

(52) U.S. Cl. 435/23

(57) ABSTRACT

Fluorogenic protease substrates are provided that comprise a peptide doubly labelled via thiol groups of the peptide with an alkyleneamidotetramethylrhodamine (alkyleneamido-TMR) group. Preferred substrates are doubly labelled with substantially pure 5-methyleneamido-TMR or 6-methyleneamido-TMR. Methods of preparing the substrates are provided, which comprise reacting the unlabelled peptide with haloalkylamido-TMR (preferably iodoacetamido-TMR). More generally, fluorogenic protease substrates are also provided which comprise a peptide doubly labelled with the same rhodamine derivative, where the two labels, and their linkages to the peptide, are substantially isomerically identical. Also provided are related methods for assaying protease activity in a sample, kits for use in such methods, and solid supports bearing the substrates of the invention.

The invention relates to fluorogenic protease substrates, more particularly to peptides doubly labelled with rhodamine-based fluorophores.

The majority of existing fluorogenic peptide protease substrates possess a C-terminal fluorescent leaving group (most commonly 7-amino-4-methylcoumarin, 7-amino-4-(trifluoromethyl)coumarin, or 7-amino-4-carbamoylmethylcoumarin). To obtain a substantial fluorescence increase (indicative of proteolytic substrate cleavage), the anilide bond which links this group to the adjacent amino acid residue must be cleaved, resulting in release of free fluorophore.

However, the utility of this class of substrates is limited since, for efficient substrate recognition, many proteases require interaction with residues on both sides of the scissile bond, as in their physiological protein substrate(s). In addition, the inclusion of additional residues flanking the residues linked by the scissile bond often enhances the selectivity of a protease substrate.

To accommodate this, a second class of fluorogenic substrates is available, which use fluorescence resonance energy transfer (FRET) to quench the fluorescence of a terminal group in the intact peptide. The general structure of the most commonly used of these is 4-(4-dimethylaminophenylazo)benzoic acid-(Xaa)ₙ-5-(2aminoethylamino)naphthalene-1-sulfonic acid (DABCYL-(Xaa)ₙ-EDANS), where (Xaa)ₙ is any amino acid sequence.

The efficiency of FRET, however, is critically dependent on the molecular distance between the donor and acceptor moieties within such a peptide, and for efficient quenching within the intact structure, n must not exceed 11-12; quenching efficiency at this distance is only about 10-fold [7].

Packard et al. [1,2] described protease assays where a peptide bearing a target sequence for a particular protease was labelled at each end with, inter alia, derivatives of tetramethylrhodamine (TMR). In their PNAS paper [1] they used carboxytetramethylrhodamine to label the N-terminus and the ε-amino group of a lysine near the C-terminus of an 11-mer peptide. In the related patent application [2] they describe alternative labelling strategies, including the option of labelling a cysteine residue at one end of the peptide with an iodoacetamidorhodamine. In a later paper by Geoghegan et al. [3] a similar strategy is employed but including the option of labelling cysteines at both peptide termini with TMR-5-maleimide.

In these latter cases, the intact, doubly labelled peptide shows an absorption spectrum characteristic of a rhodamine dimer, i.e. blue shifted compared to monomeric rhodamine. Formation of such non-covalent rhodamine dimers is well known to quench the rhodamine fluorescence. Upon cleavage by a protease specific for the particular peptide sequence, the rhodamine monomer absorption spectrum is restored and there is a concomitant fluorescence increase. The increase in fluorescence is between 3- and 15-fold [1,3]. Packard et al. [1,2] specify that so-called conformation-determining regions must be incorporated into the peptide to promote the rhodamine dimerisation, but Geoghegan et al. [3] appear sceptical of this claim.

In the work leading to the present invention, however, a 10-mer peptide (containing 8 residues from the target sequence of a malaria protease, with cysteine at each of the N— and C-termini) was labelled with 5- or 6-iodoacetamidotetramethylrhodamine (5- or 6-IATR), in particular with pure forms of the isomers. The use of this label was found to result in a dramatically larger fluorescence increase (greater than 25-fold with either isomer) on proteolytic cleavage than the previously known use of other rhodamine derivatives, e.g. TMR-5-maleimide [3].

Accordingly, in a first aspect, the present invention provides a fluorogenic protease substrate comprising a peptide doubly labelled via thiol groups of the peptide with an alkyleneamidotetramethylrhodamine (alkyleneamido-TMR) group. Thus, two alkyleneamido-TMR groups are attached to the peptide, each via a respective thiol group of the peptide.

Preferably, the alkyleneamido-TMR group is a methyleneamido-TMR group (such labelling may be accomplished, as described further below, by reaction of the peptide with haloacetamido-TMR, especially IATR). This has the advantages of high reactivity with the thiol groups of the peptide, since the halogen leaving group is located alpha to the carbonyl group, and of avoiding the production of diastereoisomeric products.

Preferably the protease substrate is doubly labelled with the same alkyleneamido-TMR group, more preferably methyleneamido-TMR (i.e. more preferably doubly labelled with methyleneamido-TMR).

Preferably the peptide is doubly labelled with a substantially pure isomeric form of the alkyleneamido-TMR group, e.g. labelled with substantially pure 5-methyleneamido-TMR or substantially pure 6-methyleneamido-TMR.

“Substantially pure alkyleneamido-TMR” in this context is intended to mean 5-alkyleneamido-TMR which is substantially pure with respect to the 6-form (and any other structural isomers), or vice versa. “Substantially pure” preferably means at least 90% pure, more preferably at least 95% pure, still more preferably at least 98%, 99%, 99.5% or 99.9% pure.

Preferably the peptide is doubly labelled with 6-alkyleneamido-TMR (more preferably 6-methyleneamido-TMR).

The fluorogenic protease substrates of the invention may be made by reacting the unlabelled peptide with haloalkylamido-TMR, preferably haloacetamido-TMR (XATR).

Accordingly, the invention further provides a method for producing a fluorogenic protease substrate comprising a peptide doubly labelled via thiol groups of the peptide with an alkyleneamidotetramethylrhodamine group, the method comprising reacting the unlabelled peptide with haloalkylamido-TMR.

Preferably the halogen atom is iodine, more preferably the XATR is iodoacetamidotetramethylrhodamine (IATR), because iodo-ATR is more reactive than other XATRs. However, bromo-ATR is thought to have a reactivity similar to that of IATR, so may also be used in the practice of the invention. Moreover, chloro-IATR may be capable of reacting with the peptide in the presence of e.g. sodium iodide (e.g. in methanol), so may similarly be used.

The production of the individual isomers of XATR, particularly IATR, is known [5,9].

As will be appreciated from the foregoing, the haloalkylamido-TMR used in the reaction is preferably substantially isomerically pure.

Generally the alkyleneamido-TMR groups will be covalently linked to the peptide via the reduced —SH side chains of cysteine residues of the peptide, the halogen atom of the haloalkanamido-TMR having acted as leaving group. This involves known chemistry, for which see the Examples and/or WO95/09170 [9] and references cited therein.

The peptide preferably contains a protease recognition sequence for a protease of interest (i.e. the amino acid motif, from a substrate of the protease of interest, that is bound by the protease). Preferably the protease recognition sequence is of from about 2 to 8, more preferably 2 to 6, still more preferably 2 to 4, most preferably 4 amino acids. Many proteases and their recognition sequences are known in the art. See for example WO96/13607 of Packard et al [2] (in which they are termed “protease binding sites”) or the study of Harris et al [15] for extensive lists of protease recognition sequences (in particular, see the contiguous non-italic residues of the peptides set out in Table 2 of WO96/13607, especially those in the four columns labelled “Protease Binding Site”). A recognition sequence in a peptide of the present invention may comprise all or part of a recognition sequence shown in one of the above references. The peptide may contain more than one protease recognition sequence for one or more proteases of interest.

While it may be generally desirable to use a peptide having a protease recognition sequence for a protease of interest, e.g. one or more of the recognition sequences listed in WO96/13607, it may also be of interest to expose the fluorogenic substrates of the invention to non-specific protease activity, e.g. using proteinase K and/or pronase. Accordingly, peptides may be used in the practice of the invention without limitation to particular protease recognition sequences.

The peptide may be of any suitable size, preferably from 4-20 amino acids in length (preferably excluding terminal cysteine residues), since peptides of this size have previously been shown to be suitable for use as fluorogenic substrates [1,3]. Preferred peptides may be from 4-15, 4-12 or 4-10 amino acids, or from 6-15, 6-12 or 6-10 amino acids.

As described in more detail below, the peptides of the present invention may lack conformation determining regions, which were thought by Packard et al [1,2] to be necessary in fluorogenic substrates of this kind. Accordingly, preferred substrates of the invention lack such a conformation determining region. Put another way, the peptide may lack conformation determining regions which bestow a generally U-shaped configuration on the peptide. Preferably, the peptide does not adopt a well-defined conformation, as determinable by NMR spectroscopy as described herein, preferably based on one or more of the following spectroscopic parameters: limited chemical shift dispersion, absence of non-sequential nOe connectivities and intermediate values (˜6 Hz) of the H_{N}—H_{α}. scalar coupling constants.

As indicated above, the alkyleneamido-TMR labels are generally attached to the peptide via the —SH side chains of cysteine residues. Preferably the peptide will include C— and N-terminal cysteine residues, for attachment of the labels. However, there is no particular need for the cysteine residues to be terminal, so labelling of internal cysteine residues is also contemplated, provided that the peptide is susceptible to protease cleavage between the cysteine residues.

Preferably the peptide contains exactly two cysteine residues.

To the best of the inventors' knowledge, very few protease recognition sequences contain a cysteine residue. By contrast, other residues to which fluorophores are conventionally attached (e.g. lysine, to which 5-carboxy-TMR is conventionally attached) may appear in protease recognition sequences, in particular for trypsin and a large number of trypsin-like proteases, which cleave peptide bonds that follow basic amino acid residues such as lysine. Accordingly, the use of thiol-linked labels, such as XATR, is particularly advantageous in that it helps to avoid interference in protease activity from labelled residues in or near the protease recognition sequence.

Labelling with the individual isomeric forms of IATR produces only a single species rather than a possible 4 different diastereoisomeric forms when the peptide is doubly labelled with TMR-5-maleimide, as in Geoghegan et al. [3], since the peptide thiol can add to either carbon in the C═C double bond of each maleimido group.

This may be responsible in part for the greater fluorescence increase shown by the inventors using IATR. Accordingly, the inventors make a general proposal that the avoidance of different isomeric forms (e.g. structural isomeric, enantiomeric and/or diastereoisomeric forms) of the substrate may lead to higher increases in fluorescence.

In a second and more general aspect, therefore, the present invention provides a fluorogenic protease substrate comprising a peptide doubly labelled with the same rhodamine derivative, where the two labels, and their linkages to the peptide, are substantially isomerically identical.

Isomerically identical is intended to mean that both instances of the label, and its linkage to the peptide, in the substrate are of the same isomeric form.

Thus, for example, where the rhodamine derivative label is capable of existing as different structural isomers (e.g. 5- and 6-IATR), both instances of the label in the substrate are of the same structural isomeric form.

Similarly, where the label is capable of existing as different stereoisomers (e.g. labels comprising a chiral carbon atom), both instances of the label in the substrate are of the same stereoisomeric form.

Moreover, different molecules of the substrate are in the same isomeric form as each other (for example, the different molecules of substrate do not form mixtures of different enantiomeric or diastereoisomeric forms).

In this context, the term “label” is intended to include both the fluorophore (i.e. the rhodamine derivative) and any group linking the fluorophore to the peptide, e.g. the acetamido group when IATR is used for labelling.

Preferably the label is linked to the peptide via thiol groups on the peptide. Suitable linkage chemistries are known in the art and include the use of a haloalkyleneamido-linking group as described above, and methanethiosulfonate linking chemistry, e.g. using the commercially available compounds T320200 (Texas Red™ 2-sulfonamidoethyl methanethiosulfonate) or S699150 (sulforhodamine methanethiosulfonate) (Toronto Research Chemicals). Further details of the latter linkage chemistry are available from the website of Toronto Research Chemicals http://www.trc-canada.com/.

Preferably the rhodamine derivative is a tetramethylrhodamine derivative. However, other rhodamine derivatives, such as Texas Red™ and tetraethylsulforhodaminerhodamine derivatives (i.e. those rhodamine derivatives present in T320200 and S699150) are also contemplated.

All preferred features of the first aspect apply, mutatis mutandis, as preferred features of this aspect also.

In a third aspect, the invention provides a method for assaying protease activity in a sample, the method comprising bringing into contact the sample and the fluorogenic substrate of either preceding aspect under conditions suitable for protease activity, and determining whether an increase in fluorescence results.

Generally, fluorescence is determined for the substrate before and after contact with the sample; since the substrate is slightly fluorescent even before proteolytic cleavage. However, the large increase in fluorescence after cleavage means that this may not be necessary.

Determination of fluorescence may be qualititative, and may even be by eye, e.g. as an indication of the presence of protease in the sample. Alternatively, determination may be quantitative, e.g. to indicate the amount or activity of protease in the sample.

Determination of fluorescence may involve comparison with stock protease solutions. The skilled person is well able to devise appropriate controls, depending on the nature of the investigation.

Preferably the step of contacting the sample and the substrate occurs at a pH of between about 5 and 10, since large fluorescence increases have been shown in this range for peptides doubly labelled with IATR. Lower pH values may lead to monomerisation of the TMR fluorophore even prior to cleavage, reducing the fluorescence increase upon cleavage.

The invention is not limited as to the nature of the sample. Indeed, it has been found that the protease substrate pepF1-R of the examples can permeate cells, so the sample may be a tissue sample, or other sample containing intact cells. In particular, the method may be a method for assaying intracellular protease activity.

Generally, the method will be for assaying activity of a known protease, and the substrate will comprise the recognition sequence for that protease. However, non-specific protease activity may be assayed using substrates not known to contain a known protease recognition sequence. Different samples of the same source may be assayed (preferably in parallel) using different substrates, for example to determine the recognition sequence specificity of a protease of unknown specificity, or to identify a protease.

In a further aspect, the invention provides a kit for use in a method of assaying protease activity, the kit comprising a fluorogenic protease substrate of the invention (preferably immobilised—see below) and a standard protease composition for calibration of the assay.

The invention described and defined herein may be regarded as a development in the field of fluorogenic protease substrates. Consequently, certain techniques and materials which may be useful in the practice of the invention have not been described herein in detail, as these are already well known to the person skilled in this field. Reference may be made in particular to WO96/13607, which is incorporated herein by reference in its entirety and for all purposes. All techniques and materials described in WO96/13607 may also be used in the practice of the present invention, insofar as they are consistent with the definitions of the invention given herein.

In particular, the detection and/or measurement of fluorescence in the practice of the present invention may be conducted as described in WO96/13607, e.g. using a fluorometer or fluorescence microscope.

The term “peptide”, as used herein, is primarily intended to mean a molecule having a plurality of naturally occurring L-amino acids, linked by peptide bonds. However, the invention is not so limited, and may also extend to molecules having one or more D-amino acids (alone, or in combination with one or more L-amino acids), particularly at positions other than those adjacent to the scissile peptide bond, or outside the protease recognition sequence. The invention may also extend to peptides including non-naturally occurring amino acids, such as α-aminoisobutryic acid, homoserine, methionine sulphoxide, methionine methylsulphonium, norleucine and hydroxyproline. Notably, norleucine may be used in place of methionine in naturally occurring protease recognition sequences, to eliminate the reactive oxidisable sulphur atom of methionine.

Moreover, the term “peptide” may also include amino acids linked otherwise than by peptide bonds, e.g. via ether linkages, particularly for bonds other than the scissile bond or bonds outside the protease recognition sequence.

The peptides of the invention may include terminal modifications, several examples of which are common in the art for various reasons, e.g. for convenience of synthesis; for example, the N-terminal amino acid may be acylated, e.g. acetylated; similarly, the C-terminal amino acid may be esterified or amidated. The presence or absence of such modification is unlikely to affect the fluorescent properties of the substrate.

The-protease substrates of the invention may be immobilised on, or modified for immobilisation on, a solid support, e.g. via a spacer region of the peptide which extends N— or C-terminally of the labelled region of the peptide.

Details of suitable spacers, supports and means of attachment are described in WO96/13607.

In a further aspect, therefore, the invention provides a solid support having immobilised thereon a fluorogenic protease substrate as defined above. Especially for parallel investigations, the invention further provides different supports respectively bearing different substrates; similarly the invention also provides a support bearing different substrates respectively immobilised at different locations (preferably discrete locations e.g. wells) of the support.

Other protease substrates, e.g. substrates intended for assaying intracellular protease activity, may be provided in combination with permeability enhancers, to assist permeation of the substrate through cell membranes. Many such permeability enhancers are known, for example the substrate may be coupled to (via a peptide linkage or otherwise) the peptide sequence Penetratin (WO91/18981).

### EXAMPLE 1

### Fluorogenic Substrate Based on a Malarial Serine Protease Recognition Sequence

Summary

Merozoites of the human malaria parasite Plasmodium falciparum invade and replicate within red blood cells. Invasion is known to require the activity of parasite serine proteases. PfSUB-1 is a subtilisin-like serine protease expressed in the P. falciparum merozoite, and its function is presently under investigation. Enzymatically active PfSUB-1 has been produced in a recombinant form using the baculovirus/insect cell system [4]. Immediately following translation, PfSUB-1 undergoes an autocatalytic activation step in which the pro-enzyme is cleaved at an internal Asp-Asn bond within the motif ²¹⁵LVSAD↓NIDIS²²⁴. We found that an N-acetylated synthetic decapeptide (Ac-LVSADNIDIS—OH) based on this site is cleaved at the Asp-Asn bond by both recombinant PfSUB-1 and authentic parasite-derived enzyme [4]. To adapt this peptide substrate for use in a fluorescence-based assay, the derivative Ac-CVSADNIDIC—OH was alkylated at both cysteine side-chains with 6-iodoacetamidotetramethylrhodamine (6-IATR) to produce an internally quenched substrate. Cleavage of this compound results in a large increase in fluorescence. We have begun to analyse in detail the biophysical properties of the substrate. In addition, the compound has been used to develop a simple and robust microtitre plate-based fluorescence assay to measure enzyme activity, and is suitable for adaptation to high throughput format.

Experimental Methods and Results

Synthesis of the Fluorogenic Peptide pepF1-R.

Synthesis of Ac-CVSADNIDIC—OH was by standard solid phase Fmoc chemistry. Prior to conjugation with 6- or 5-iodoacetamidotetramethylrhodamine (6-IATR or 5-IATR) [5,6], the peptide was first treated with the reducing agent tris(2-carboxyethyl)phosphine (TCEP) to ensure complete accessibility of the cysteine side-chain sulfhydryls. 10 mg of peptide was dissolved in 910 μl dimethylformamide and added to 8.2 ml reduction buffer (50 mM Tris-HCl pH 7.6). TCEP was then added from a freshly-prepared 40 mM stock in water to a final concentration of 2 mM (thus at a two-fold molar excess over peptide). The mixture was incubated under nitrogen at 21° C. for 18 hours, then the reduced peptide was purified by RP-HPLC on a Vydac 10 mm×25 cm semi-preparative C₁₈ column, eluting at 4.7 ml min⁻¹ with a 4.5-31.5% (v/v) gradient of acetonitrile in 0.1% TFA.

The reduced peptide was lyophilised, taken up in 910 μl dimethylformamide, added to 7.3 ml reduction buffer containing 0.1 mM EDTA, then immediately supplemented with 910 μl 6-IATR stock (21.3 mM in dimethylformamide), stirring continuously. After a further overnight incubation under nitrogen in the dark at room temperature, the reaction was quenched by the addition of 280 μl 2M sodium 2-mercaptoethanesulfonate (MESNA). Following a further 2 h incubation at room temperature, the crude reaction mixture was applied to a G10 Sephadex column (1.6×65 cm) equilibrated in 30% (v/v) acetic acid, and eluted at a rate of 4 ml h⁻¹, collecting 4 ml fractions. Samples (20 μl) of individual fractions were analysed on a Vydac 4.6 mm×25 cm C₁₈ reversed-phase column, eluted at 1 ml min⁻¹ with a 4.5-58.5% (v/v) gradient of acetonitrile in 0.1% TFA. Samples of the digestion products were collected manually, dried under vacuum, dissolved into 60% (v/v) acetonitrile, 0.1% formic acid and analysed by electrospray mass spectrometry on a Micromass Platform single quadrapole mass spectrometer (Micromass UK Ltd., Altringham, UK). The first peak to elute from the G10 column contained predominantly the doubly labelled peptide (observed mass 1,978.0 Da, calculated mass 1,976.9 Da).

The relevant G10 Sephadex fractions were pooled and purified on a Vydac 10 mm×25 cm semi-preparative C₁₈ RP-HPLC column, eluting at 4.7 ml min⁻¹ with a 22.5-36% (v/v) gradient of acetonitrile in 0.1% TFA. The purified compound (subsequently referred to as pepF1-R) was lyophilised, taken up in DMSO, and stored over dessicant in the dark at −20° C.

For use in NMR measurements, the compound was dissolved directly into deuterated DMSO.

A similar protocol was used for the preparation of peptide doubly labelled with 5-IATR.

NMR Spectroscopy

NMR spectra were recorded at proton frequencies of 800 MHz and 500 MHz on Varian Inova and Unityplus spectrometers respectively. Spectra of pepF1-R were recorded in 10% CD₃OD/90% H₂O at 25° C. The low solubility in this solvent system limited the sample concentration to 50 μM and necessitated the use of the highest available field. Two-dimensional TOCSY [10] and NOESY [11] spectra were recorded, employing WATERGATE [12] for solvent suppression. Quadrature detection in indirect dimensions was achieved using the States procedure [13]. Spectra were referenced with respect to the residual water resonance at 4.75 ppm. Spectra were processed and analysed using the nmrPipe/nmrDraw package [14]. Molecular modelling was performed using InsightII (Accelrys Inc). Control 1-dimensional nOe spectra of 6-chloroacetamidotetramethylrhodamine [5] were obtained for a 2 mM solution in CDCl₃—MeOH-d4 (7:3 v/v) at 800 MHz.

The complete NOESY spectrum of pepF1-R, together with the 2-D TOCSY spectrum, revealed spin systems for the 10 amino acids. Notably, chemical shift differences were either undetectable or very small for structurally comparable protons on the two rhodamines, suggesting that two dye moieties on the labeled peptide were in similar environments. Several spectroscopic parameters indicate that the peptide does not adopt a single well-defined conformation: these were the limited chemical shift dispersion, absence of non-sequential nOe connectivities and intermediate values (˜6 Hz) of the H_{N}—H_{α} scalar coupling constants. nOe cross peak volumes were used to derive approximate inter-proton distance restraints for the rhodamines, using a simple r⁻⁶ relationship and corrected for the number of protons contributing to each cross peak.

Absorption Spectroscopy

All dye concentrations were determined from the absorption at 528 nm, with ε 52,000 M⁻¹cm⁻¹ [5]. Solutions of pepF1-R and its isomer derived from 5-IATR were prepared at ˜4 μM by dilution from stock DMSO solutions into digestion buffer (20 mM Tris, 50 mM NaCl, 12 mM CaCl₂, 0.05% NP-40 (w/v), pH 7.6). It was necessary to include NP-40 detergent (Boehringer Mannheim) in all buffers containing the labelled peptides to avoid non-specific adsorption to glass and plasticware. Portions (4 ml) of each solution were treated with an aliquot of pronase (16 μl of 1 mg ml⁻¹ in the same buffer) and incubated at room temperature for 30 min. Absorbance spectra of these solutions and of the corresponding labelled peptides prior to pronase treatment were recorded (Beckman DU640 spectrophotometer).

Multi-Well Fluorescence Assay

Purified recombinant PfSUB-1 at a range of concentrations in digestion buffer was dispensed into 50 μl aliquots in wells of white 96-well microtitre plates (FluoroNunc, NUNC). Wells were supplemented with either 0.5 μl 100 mM p-hydroxymercuribenzoate (pHMB, a potent inhibitor of PfSUB-1 [4]) or 0.5 μl water, then the plates were placed on a rotary shaker and mixed at room temperature for 30 min prior to the addition of 50 μl of a solution of pepF1-R at various concentrations in digestion buffer. Plates were sealed and incubated at 37° C. for 18 h before being read on a Perkin Elmer LS-50B Luminescence spectrometer fitted with a LS50B WPR multi-plate reader accessory. Measurements were performed at an excitation wavelength of 552 nm, slit width 4 nm, and an emission wavelength of 580 nm, slit width 2.5 nm. Data collection was managed using Perkin Elmer FL WinLab software.

The fluorescence increase was proportional to the concentration of protease used, and was virtually completely ablated in the presence of the PfSUB-1 inhibitor pHMB.

Initial Characterisation of pepF1-R

For protease digestion experiments, pepF1-R was diluted directly from DMSO stocks into digestion buffer (20 mM Tris-HCl pH 7.6, 50 mM NaCl, 12 mM CaCl₂, ±0.05% w/v Nonidet P40). The DMSO concentration in digestion experiments was maintained below 1% (v/v).

Incubation of pepF1-R with pronase (Boehringer Mannheim) or purified recombinant PfSUB-1 [4] resulted in a dramatic, time-dependent increase in fluorescence (fluorescence measurements were performed at an excitation wavelength of 552 nm, slit width 4 nm, and an emission wavelength of 580 nm, slit width 2.5 nm). Incubation with PfSUB-1 which had been pre-treated with 1 mM p-hydroxymercuribenzoate, a potent inhibitor of the enzyme [4], resulted in no fluorescence increase.

A sample of pepF1-R digested with PfSUB-1 was subjected to RP-HPLC-fractionation (Vydac 4.6 mm×25 cm C₁₈ reversed-phase column, eluted at 1 ml min⁻¹ with a 4.5-58.5% (v/v) gradient of acetonitrile in 0.1% TFA). In addition to a small peak at 38.5 min corresponding to residual pepF1-R, two large new fluorescent peaks of equal height were evident, with retention times of 29.6 and 34.0 min. Analysis of these by mass spectrometry showed that they corresponded to the expected products of cleavage at the Asp-Asn bond; the observed masses of the products were 977.5 Da (corresponding to the N-terminal cleavage product, calculated mass 977.5 Da) and 1018.4 Da (corresponding to the C-terminal cleavage product, calculated mass 1018.5 Da).

Spectrometric analysis of intact pepF1-R and its 5-IATR-labelled isomer showed that each possesses an absorption spectrum characteristic of dimeric tetramethylrhodamine [1-3,5]. For these measurements, the compound was diluted 200-fold from a 230 μM stock solution in DMSO into 20 mM Tris-HCl pH 7.6. Identical spectra were obtained upon dilution into 20 mM acetate buffer, pH 5.0, or 20 mM ethanolamine pH 10.0. All buffer solutions contained 0.05% NP-40 detergent (w/v). Addition of recombinant PfSUB-1 or pronase to the pH 7.6 solution resulted in a time-dependent shift towards a spectrum characteristic of the rhodamine monomer, concomitant with a greater than 25-fold increase in fluorescence.

Discussion

In concordance with the findings of Geoghegan et al. [3] this work shows that it is unnecessary to incorporate “conformation determining” regions into the intervening peptide sequence in order to promote efficient interaction of the rhodamine monomers. Indeed, NMR data indicate that the peptide sequence in pepF1-R is quite unstructured. This is important, since it suggests that there are essentially no restrictions on the peptide sequence that may be used, in turn indicating that this approach has wide application for protease substrate design.

Moreover, substrates based on the approach reported here are not subject to the same size constraints as the FRET-based substrates referred to earlier. This is an important consideration when the precise recognition requirements of a protease are unknown. Furthermore, synthesis of the doubly labelled peptide is relatively simple, due to the requirement only to incorporate an identical group at each of the two available reactive side-chain; synthesis of FRET-based substrates is much more labour-intensive, generally requiring a solid-phase step [8].

The pepF1-R substrate can be used in a microtitre scale assay suitable for scale-up to high throughput format, allowing screening of large libraries of potential inhibitors. Our work shows that interaction of the IATR monomers is efficient between pH 5 and 10, allowing the substrates of the invention to be used for analysing the pH dependence of protease (e.g. PfSUB-1) activity over at least this range.

PepF1-R, and other substrates of the invention, may also have applications for exploring the activity of PfSUB-1 in situ in the malaria merozoite, where the protease accumulates in secretory granules; preliminary experiments suggest that pepF1-R is readily membrane-permeable, and cleavage of the intracellular compound may allow visualisation of these organelles by fluorescence microscopy.

References

[1] Packard et al (1996) Proc. Natl. Acad. Sci. USA 93, 11640.

[2] WO96/13607.

[3] Geoghegan et al (2000) Bioconjugate Chem. 11, 71.

[4] Sajid et al (2000) J. Biol. Chem. 275, 631.

[5] Corrie and Craik (1994) J. Chem. Soc., Perkin Trans. 1, 2967.

[6] Sabido-David et al (1998) Biophys. J. 74, 3083.

[7] Holskin et al (1995) Anal. Biochem. 227, 148.

[8] Grahn et al (1998) Anal. Biochem. 265, 225.

[9] WO95/09170

[10] Braunschweiler and Ernst (1983) J. Magn. Reson. 53, 521-528.

[11] Jeener et al (1979) J. Chem. Phys. 71, 4546-4553.

[12] Piotto et al (1992) J. Biomol. NMR 2, 661-665.

[13] States et al (1982) J. Magn. Reson. 48, 286-292.

[14] Delaglio et al (1995) J. Biomol. NMR 6, 277-293.

[15] Harris et al (2000) Proc. Natl. Acad. Sci. U.S.A. 97, 7754-7759.

1. A fluorogenic protease substrate comprising a peptide having thiol groups and being doubly labelled via said thiol groups with an alkyleneamidotetramethylrhodamine (alkyleneamidoTMR) group.

2. A fluorogenic protease substrate according to claim 1, which is doubly labelled with the same alkyleneamido-TMR group.

3. A fluorogenic protease substrate according to claim 2, wherein the alkyleneamido-TMR group is a methyleneamido-TMR group.

4. A fluorogenic protease substrate according to claim 2, wherein the peptide is doubly labelled with an isomeric form of the alkyleneamido-TMR group that is at least 90% pure with respect to other isomeric forms of the alkyleneamido-TMR group.

5. A fluorogenic protease substrate according to claim 4, wherein the alkyleneamido-TMR group is 5-alkyleneamido-TMR or 6-alkyleneamido-TMR.

6. A fluorogenic protease substrate according to claim 4, wherein the level of purity is at least 95%.

7. A fluorogenic protease substrate according to claim 6, wherein the level of purity is at least 98%.

8. A fluorogenic protease substrate according to claim 1, which contains one or more protease recognition sequences for one or more proteases of interest.

9. A fluorogenic protease substrate according to claim 8, wherein the protease recognition sequence is 2 to 8 amino acids in length.

10. A fluorogenic protease substrate according to claim 1, which is 4-20 amino acids in length, optionally excluding any terminal cysteine residues.

11. A fluorogenic protease substrate according to claim 10, which is 4-12 amino acids in length.

12. A fluorogenic protease substrate according to claim 11, which is 6-10 amino acids in length.

13. A fluorogenic protease substrate according to claim 1, which does not adopt a well-defined conformation, as determinable by NMR spectroscopy.

14. A fluorogenic protease substrate according to claim 1, wherein the alkyleneamido-TMR groups are attached to the peptide via cysteine residues.

15. A fluorogenic protease substrate according to claim 14, wherein the cysteine residues are C— and N-terminal cysteine residues.

16. A fluorogenic protease substrate according to claim 14, wherein the cysteine residues are internal, and the peptide is susceptible to protease cleavage between the cysteine residues.

17. A fluorogenic protease substrate according to claim 1, wherein the peptide contains exactly two cysteine residues.

18. A method for producing a fluorogenic protease substrate as claimed in claim 1, the method comprising reacting an unlabelled peptide containing two thiol groups with haloalkylamido-TMR.

19. A method according to claim 18, wherein the halogen atom of the haloalkylamido-TMR is iodine.

20. A method according to claim 19, wherein the haloalkylamido-TMR is iodoacetamidotetramethylrhodamine (IATR).

21. A fluorogenic protease substrate comprising a peptide doubly labelled with the same rhodamine derivative, wherein the two labels, and their linkages to the peptide, are substantially isomerically identical.

22. A fluorogenic protease substrate according to claim 21, wherein the label is linked to the peptide via thiol groups on the peptide.

23. A fluorogenic protease substrate according to claim 21, wherein the rhodamine derivative is a tetramethylrhodamine derivative.

24. A method for assaying protease activity in a sample, the method comprising bringing into contact the sample and a fluorogenic substrate, as claimed in claim 1 under conditions suitable for protease activity, and determining whether an increase in fluorescence results.

25. A method according to claim 24, wherein fluorescence is determined for the substrate before and after contact with the sample.

26. A method according to claim 24, wherein the step of contacting the sample and the substrate occurs at a pH of between about 5 and 10.

27. A method according to claim 24, wherein the sample contains intact cells.

28. A method according to claim 24, wherein activity of a known protease is assayed, and wherein the substrate comprises the recognition sequence for that protease.

29. A kit for use in a method of assaying protease activity, the kit comprising a fluorogenic protease substrate as claimed in claim 1 and a standard protease composition for calibration of the assay.

30. A kit according to claim 29, wherein the fluorogenic protease substrate is immobilised.

31. A solid support having immobilised thereon a fluorogenic protease substrate as claimed in claim 1.

32. A solid support according to claim 31, bearing different said substrates respectively immobilised at different locations of the support.

33. A method according to claim 27, wherein said sample is a tissue sample.
